(19) United States
(12) Patent Application Publication
(10) Pub. No.: US 2010/0310674 A1
(43) Pub. Date: Dec. 9, 2010

(54) NOVEL COMPOSITION FOR TREATING THE SIDE EFFECTS OF ANTICANCER TREATMENTS

(75) Inventors: Rebecca Pruss, Cassis (FR); Thierry Bordet, Paris (FR); Jean-Louis Abitbol, Antony (FR); Antoine Beret, Marseille (FR)

Correspondence Address: BROWDY AND NEIMARK, P.L.L.C.;624 NINTH STREET, NW, SUITE 300, WASHINGTON, DC, 20001-5303, US

(73) Assignee: TROPHOS, Marseille Cedex 9 (FR)

(21) Appl. No.: 12/740,478

(22) PCT Filed: Oct. 30, 2008
(86) PCT No.: PCT/FR08/01528

§ 371 Date: Aug. 23, 2010

(30) Foreign Application Priority Data
Oct. 30, 2007 (FR) 07 07625

(51) Int. Cl. A61K 33/24; A61K 31/575; A61P 35/00; A61P 25/00

(52) U.S. Cl. 424/649; 514/171

(57) ABSTRACT

The invention relates to novel compositions, particularly pharmaceutical, comprising, as active ingredients, at least one cytotoxic agent and at least one cholest-4-en-3-one oxime derivative.

The invention relates to novel pharmaceutical compositions. More particularly, according to the invention, pharmaceutical compositions are provided comprising as active ingredients at least one cytotoxic agent and at least cholest-4-en-3-one oxime or one of its derivatives.

The main medicaments used in anticancer chemotherapy can be classified according to their activity. Thus the following are defined:
cytotoxic agents;
hormones and/or agents blocking the secretion of hormones or antagonists;
immune response modifiers;
monoclonal antibodies.

Among these agents, the cytotoxic agents are used in order to destroy all cancerous cells.

Most cytotoxic agents interact with cellular DeoxyriboNucleic Acid (DNA) or its precursors: they inhibit DNA synthesis or induce irreparable DNA lesions. Certain cytotoxic agents act after the transcription phase: they interact with proteins and enzymes involved in cell proliferation/division. Cytotoxic medicaments are not specific to cancerous cells, they also act on normal cells. It is this non-specificity that results in their toxicity.

Moreover, in the present text, by the term “cytotoxic agent” is meant a cytotoxic agent, advantageously an anticancer agent, possibly inducing in the patient symptoms of peripheral neuropathies and/or damage to the nervous system.

It is in fact well known that one of the main difficulties with chemotherapy of malignant tumours lies in the fact that all the cytotoxic agents which prove to be capable of inhibiting the proliferation of tumour cells exhibit toxic side effects vis-à-vis normal cells.

Thus, haematological, gastro-intestinal, dermatological toxicity, delayed growth in children, gonadal toxicity, cardiac, pulmonary, hepatic toxicity, nephrotoxicity, vesical toxicity, ototoxicity, toxicity to the epithelial tissues (veins and mucous membranes), or also neurotoxicity such as, for example, peripheral neuropathies are described.

By peripheral neuropathies is meant within the meaning of the invention, different medical diseases of the peripheral nerve, excluding impairments of the anterior horn of the spinal cord. The peripheral neuropathies are divided into three major groups:
mononeuropathies defined by the impairment of a single nerve trunk;
multiple mononeuropathies characterized by successive impairment of several nerve trunks;
and distal polyneuropathies with diffuse and symmetrical impairment of the four limbs.

The clinical signs include muscle atrophy, cramps, fasciculations and motor dysfunction, anaesthesia, tactile or proprioceptive hypoesthesia, ataxia, hypoalgesia, dolorific anaesthesia, thermal hypoesthaesia, paresthesias, dysaesthesias, hyperpathia, hyperalgesia, allodynia, areflexia, hyporeflexia (Stojkovic; La revue de médecine interne 27, (2006) 302-312),

Peripheral neuropathies can be, inter alia, caused by the cytotoxic agents used in anticancer treatments.

These peripheral neuropathies can sometimes disappear when the administration of said medicaments is stopped. However it sometimes takes several weeks, or even months, before any improvement is observed.

To the knowledge of the Applicant and without disparaging current knowledge there is no satisfactory treatment against the symptoms of peripheral neuropathies or the damage caused to the nervous system by cytotoxic agents, particularly anticancer cytotoxic agents, whether treatment is combined with said anticancer treatment, in the form of a single composition comprising the cytotoxic agent and the agent treating the symptoms of peripheral neuropathies and/or the damage caused to the nervous system by said agents or in the form of different compositions administered simultaneously or successively. The undesirable side effects of cytotoxic agents lead in particular to a limitation in the main treatment and in the expected benefit (Wickham, Clinical Journal of Oncology Nursing, 11, (3), (2007), 361-376). For example, it is sometimes necessary to reduce the doses of anticancer agents at the time of administration, or to administer lower doses more frequently, to infuse over longer periods, or to space out the administrations, or also to stop treatment with an agent in the case of peripheral neuropathies or in order to reduce the risks of peripheral neuropathies.

A need therefore exists in the field of the treatment and/or prevention of the neuropathies induced by cytotoxic agents, particularly in their use as anticancer agents.

It is one of the objectives of the present invention to provide a composition intended for the treatment of cancer, which at the same time prevents and/or treats the peripheral neuropathies which are an undesirable and limiting side effect of the cytotoxic agent used for the treatment of cancer.

The composition according to the invention has been developed for this purpose as a function of the following criteria:
reduction of side effects of the “peripheral neuropathies” type caused by the cytotoxic agent;
cytotoxic activity of said agent, particularly of said anticancer agent, used unchanged, or even improved because, due to the reduction in the side effects, the anticancer treatment can take longer and/or the doses administered can be greater.

The present invention is a response to this demand. It offers decisive advantages. In fact, the novel pharmaceutical compositions of the invention exhibit a cytotoxic activity linked to the quantity of cytotoxic agent present in the composition and delay the appearance of the peripheral neuropathies and/or reduce the sensation of pain induced by said cytotoxic agent used.

In fact, unexpectedly and surprisingly the Applicant has found that the peripheral neurotoxicity of the cytotoxic agents, particularly those used as anticancer agents, can be considerably reduced when said cytotoxic agents are administered to the organism in the form of a composition comprising said cytotoxic agent and at least cholest-4-en-3-one oxime or a compound of the cholest-4-en-3-one oxime family.

Thus, a first subject of the invention is a composition, particularly a pharmaceutical composition, comprising at least one cytotoxic agent, particularly an anticancer cytotoxic agent and/or an agent inducing symptoms of peripheral neuropathies and/or damage to the nervous system in the patient, preferentially an anticancer cytotoxic agent inducing symptoms of peripheral neuropathies and/or damage to the nervous system in the patient and at least cholest-4-en-3-one oxime or one of its derivatives.

A surprising aspect of the invention resides in the fact that the reduction in peripheral neurotoxicity has been shown with different anticancer agents, of which it is known that their action mechanism vis-à-vis cancer is different, as well as their mechanism leading to the appearance of peripheral neuropathies (Wickham, Clinical Journal of Oncology Nursing, 11, (3), (2007), 361-376).

An advantageous aspect of the invention resides in the fact that said pharmaceutical composition makes it possible to prevent and/or treat peripheral neuropathies without modifying the efficacy of the cytotoxic agent.

According to the invention, the composition can comprise as cytotoxic agent at least one anticancer cytotoxic agent, possibly having peripheral neuropathies as an undesirable side effect. It is understood that the composition according to the invention can comprise as cytotoxic agent, several particularly anticancer cytotoxic agents, and/or cytotoxic agents having peripheral neuropathies as an undesirable side effect, and very particularly anticancer cytotoxic agents having peripheral neuropathies as an undesirable side effect, or also a mixture of at least one such agent with at least one other cytotoxic agent, optionally anticancer but not having peripheral neuropathies as an undesirable side effect.

Preferably according to the invention, the cytotoxic agent included in the composition according to the invention can be an anticancer cytotoxic agent which may or may not have peripheral neuropathies as an undesirable side effect, and very advantageously an anticancer cytotoxic agent having peripheral neuropathies as an undesirable side effect.

Similarly, it is understood that the composition, particularly the pharmaceutical composition, according to the invention, can comprise one or more compounds of the cholest-4-en-3-one oxime family.

As cytotoxic agent, particularly anticancer cytotoxic agent known to cause peripheral neuropathies, there may particularly be mentioned the platinum derivatives, in particular cisplatin and its derivatives, such as carboplatin and oxaliplatin, periwinkle alkaloids (vinca alkaloids) in particular vinblastine, vincristine, vindesine or also vinorelbine, or the taxanes, such as paclitaxel or docetaxel.

These agents are preferably used according to the invention. As indicated previously, these agents can be used according to the invention, alone or combined with each other or combined with another cytotoxic agent, particularly an anticancer cytotoxic agent, very particularly with at least one other cytotoxic agent, particularly another anticancer cytotoxic agent not having peripheral neuropathies as a side effect.

An effect as sought can be obtained when the composition according to the invention also comprises as active ingredient one or more cytotoxic agents, at least one compound corresponding to formula I, particularly cholest-4-en-3-one oxime.

These compounds of formula I, particularly cholest-4-en-3-one oxime and their derivatives which reduce or treat peripheral neuropathies, are described in international applications WO 2004/082581 and WO 2007/080270.

Thus, the invention relates to a composition, particularly a pharmaceutical composition, characterized in that it comprises at least:

a compound of formula I

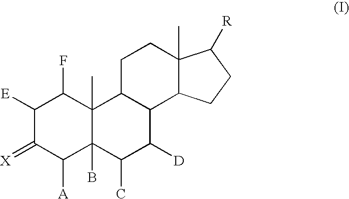

in which

X represents an ═N—OH group,

R represents a group chosen from

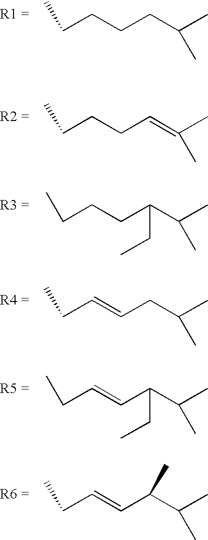

A represents a hydrogen atom or together with B a carbon-carbon bond,

B represents a hydrogen atom, a hydroxy group or together with A a carbon-carbon bond,

C represents a hydrogen atom, a ketone group or an oxime group (=NOH), or together with D a carbon-carbon bond,

D represents a hydrogen atom or together with C a carbon-carbon bond,

E represents a hydrogen atom or together with F a carbon-carbon bond,

F represents a hydrogen atom or together with E a carbon-carbon bond,

or one of its addition salts with pharmaceutically acceptable acids, or one of its esters or one of the addition salts with the pharmaceutically acceptable acids of said esters and at least one cytotoxic agent, particularly an anticancer cytotoxic agent.

The addition salts with pharmaceutically acceptable acids can be for example salts formed with hydrochloric, hydrobromic, nitric, sulphuric, phosphoric, acetic, formic, propionic, benzoic, maleic, fumaric, succinic, tartaric, citric, oxalic, glyoxylic, aspartic or alkane sulphonic acids such as methane or ethane sulphonic, or arylsulphonic acids, such as benzene or paratoluene sulphonic, or carboxylic acids.

By esters is meant according to the invention the oxime esters which can be prepared by techniques known to a person skilled in the art, for example by an esterification reaction between an acid and the hydroxy-imine (or oxime). As examples of oxime esters, there may be mentioned acetates, proprionates, oxalates, succinates, tartrates, fumarates, malonates, or also methanesulphonates, ethanesulphonates or also phosphates. The term oxime esters can also represent esters which can be hydrolyzed in vivo for example by metabolic route.

It is understood according to the invention that the oxime group represents the two syn and anti isomers in a mixture or either one separately.

Advantageously, according to the invention at least one compound of formula I is used, chosen from the compounds for which, X representing an oxime group (═NOH) then:
A represents together with B a carbon-carbon bond, C, D represent a hydrogen atom, E, F represent a hydrogen atom or together a carbon-carbon bond and R has the meaning R1,
A represents together with B a carbon-carbon bond, C, D represent a hydrogen atom, E, F represent a hydrogen atom and R has the meaning R2 or R3 or R4,
A represents together with B a double bond, C represents together with D a carbon-carbon bond, E, F represent a hydrogen atom and R has the meaning R1 or R6,
A represents together with B a double bond, C represents together with D a carbon-carbon bond, E represents together with F a carbon-carbon bond and R has the meaning R1,
E represents together with F a double bond, C, D, A, B represent a hydrogen atom, and R has the meaning R1,

or one of its addition salts with pharmaceutically acceptable acids, or one of its esters or one of the addition salts with the acceptable acids of said esters,

Still more advantageously according to the invention, the compound of formula I can be chosen from cholestan-3-one oxime, cholest-4-en-3-one oxime, cholest-1,4-dien-3-one oxime, very preferably cholest-4-en-3-one oxime or cholest-1,4-dien-3-one oxime, or one of their addition salts with pharmaceutically acceptable acids, or one of their esters or one of the esters of the addition salts.

Very preferably the compound of formula I used according to the invention is cholest-4-en-3-one oxime.

It is understood according to the invention that the oxime group represents the two syn and anti isomers in a mixture or isolated.

According to the invention the cytotoxic agent, particularly the anticancer cytotoxic agent, very particularly the agent having peripheral neuropathies as an undesirable side effect can be an agent chosen from all the known cytotoxic agents, particularly the anticancer cytotoxic agents, more particularly the agents having peripheral neuropathies as an undesirable side effect. There may be mentioned particularly cisplatin and its derivatives, such as carboplatin and oxaliplatin, periwinkle alkaloids (vinca), such as vinblastine, vincristine, vindesine and vinorelbine, or also the taxanes, such as docetaxel and paclitaxel.

In a preferred manner, oxaliplatin, carboplatin, cisplatin, vinblastine, vincristine or paclitaxel can be used according to the invention.

Very preferably, according to the invention, oxaliplatin, vincristine or paclitaxel will be used.

The term “cytotoxic agent having peripheral neuropathies as an undesirable effect” designates any cytotoxic agent, particularly any anticancer cytotoxic agent which, during treatment, causes peripheral neuropathies in the patient.

The composition according to the invention can be presented in different forms such as for example a single composition comprising the different compounds used according to the invention, but also in the form of two or more compositions each comprising at least one of the different compounds used according to the invention, whether said compositions are administered simultaneously or successively.

Preferentially according to the invention, the composition will be understood as a single composition comprising at least the two compounds used according to the invention.

By “pharmaceutical composition”, is meant in the present invention, a composition the components of which are pharmaceutically acceptable. For example, when oral administration is envisaged, the components are appropriate or acceptable for oral administration.

In the composition according to the invention, the compounds can be advantageously present in physiologically effective doses.

Thus, for example, according to an embodiment of the invention, the cytotoxic agent and/or cholest-4-en-3-one oxime or one of its derivatives can each be in the composition in a quantity comprised between 0.1 and 2000 mg/ml.

According to another embodiment of the invention, the cytotoxic agent and/or cholest-4-en-3-one oxime or one of its derivatives can be in the composition in a cytotoxic agent/cholest-4-en-3-one oxime or derivatives ratio comprised between 0.1 and 300.

Finally according to another preferred embodiment, the cytotoxic agent and/or cholest-4-en-3-one oxime or one of its derivatives can each be in the composition in a quantity comprised between 0.1 and 2000 mg/ml and in a cytotoxic agent/cholest-4-en-3-one oxime or derivatives ratio comprised between 0.1 and 300.

Among the compositions according to the invention, there may be mentioned as preferred compositions a composition comprising cholest-4-en-3-one oxime and oxaliplatin, a composition comprising cholest-4-en-3-one oxime and vincristine or also a composition comprising cholest-4-en-3-one oxime and paclitaxel.

The composition according to the present invention can be used in mammals, more precisely in humans.

According to the invention, the compounds of the composition can be in a common galenic form.

Also according to the invention, each of the compounds of the composition can be presented in a galenic form identical to or different from that of the other compound.

It is therefore understood that when, according to the invention, the compounds of the composition are in a common galenic form, they are administered simultaneously and by the same administration route.

It is also understood that when, according to the invention, each of the compounds of the composition is in a galenic form identical to, or different from, that of the other compound, they can be administered either simultaneously or successively, by identical or different administration routes.

Preferably, the cytotoxic agent can be administered daily or with a time lapse of less than or more than a day between two administrations, this time lapse being able to vary over the course of the treatment. In a preferred manner, the compound of formula I can be taken daily, before, during or after an administration of anticancer agent, or before, during and after, or also, before and during, before and after, or during and after an administration of the cytotoxic agent. It can also be envisaged that the cytotoxic agent is changed during the course of the treatment of the same patient, in order to obtain an optimized anticancer effect.

As medicaments, cholest-4-en-3-one oxime or one of its derivatives, particularly the compounds corresponding to formula I, their esters, their addition salts with the pharmaceutically acceptable acids as well as the addition salts with the pharmaceutically acceptable acids of said esters can be formulated for the digestive or parenteral route.

The medicaments according to the invention can moreover comprise at least one other therapeutically active ingredient, whether active on the same pathology or on a different pathology, for simultaneous or separate use or use spread over time, in particular during treatment of a subject suffering from cancer.

According to the invention, the composition can moreover comprise one (or more) excipient(s) or inert physiologically acceptable vehicle(s), i.e. pharmaceutically inactive and non-toxic. There can be mentioned for example, saline, physiological, isotonic, buffered solutions, etc., compatible with a pharmaceutical use and known to a person skilled in the art. The compositions according to the invention can contain one or more agents or vehicles chosen from dispersants, solubilizers, stabilizers, preservatives, etc.

The compositions according to the invention can be formulated in the form of an injectable suspension, gels, oils, tablets, suppositories, powders, gelatin capsules, capsules, etc., optionally using galenic forms or devices ensuring sustained and/or controlled release. For this type of formulation, an agent such as cellulose, carbonates or starches are advantageously used.

It can be envisaged that one of the active compounds of the composition according to the invention is formulated in an injectable solution, whereas the other or others is/are in, for example, a gelatin capsule form for oral administration.

The administration can be carried out by any method known to a person skilled in the art, preferably by oral route or by injection, typically by intra-peritoneal, intracerebral, intrathecal, intravenous, intra-arterial, intramuscular or subcutaneous route. In preferred manner, the anticancer agent is administered by intravenous route and the compound of formula I is administered by oral route.

For the injections, the compounds are generally packaged in the form of liquid suspensions, which can be injected using syringes or infusions, for example. It is understood that the flow rate and/or the injected dose, or the dose to be administered generally, can be adapted by a person skilled in the art depending on the patient, the pathology, the administration method, etc. It is understood that repeated administrations can be carried out, optionally in combination with other active ingredients and/or any pharmaceutically acceptable vehicle (buffers, saline, isotonic, solutions in the presence of stabilizers, etc.).

The invention can be used in mammals, in particular in humans.

In general, the daily doses of the compounds are minimum doses for obtaining the desired therapeutic effect. The doses will depend on many factors, in particular the administration route, the duration of administration, the time of administration, the rate of elimination of the compound, the different product or products used in combination with the compound, the age, weight and physical condition of the patient, as well as their medical history, and any other known medical information.

The doses of the anticancer compounds and compounds of formula I and for example of cholest-4-en-3-one oxime are in general comprised between 0.001 to 100 mg per kilo per day for humans.

If necessary, the daily dose can be administered in two, three, four, five, six or more administrations per day, or by multiple sub-doses administered at appropriate intervals during the day.

It is also understood that the anticancer treatment can correspond to a non-daily administration, as is usually the case with the anticancer compounds of the invention.

A subject of the invention is also the use of the composition according to the invention for the manufacture of a cytotoxic medicament intended to treat cancer while preventing and/or treating the side effects of peripheral neuropathy type caused by the anticancer treatment.

A subject of the invention is also the use of the composition according to the invention for the manufacture of a cytotoxic medicament intended to prevent and/or treat the side effects of peripheral neuropathy type caused by the anticancer treatment.

Another subject is a method for preventing the appearance of side effects of peripheral neuropathy type during anticancer treatments, by using a composition according to the invention.

A subject of the invention is also the use of at least cholest-4-en-3-one oxime or one of its derivatives, particularly a compound of formula I, in combination with a cytotoxic agent as described previously for the manufacture of a medicament intended to improve the side effects of an anticancer treatment.

A subject of the invention is also a pharmaceutical kit for the treatment of cancer, including at least one cytotoxic anticancer compound and at least one compound of formula I.

The following examples illustrate the present application.

### EXAMPLE 1

### Effect of cholest-4-en-3-one oxime on the Neuropathies Induced by Vincristine Sulphate

The effect of cholest-4-en-3-one oxime on tactile allodynia induced by cumulative doses of vincristine sulphate was evaluated in the rat.

Methods

Adult male WISTAR rats weighing approximately 250 g were used. Vincristine sulphate was injected by intravenous route at 200 μg/kg on days 1, 4 and 6 (total cumulative dose of 600 μg/kg). The animal's paw withdrawal thresholds to harmless mechanical stimulation were measured in the rats on days 0, 3, 5, 7 and then every day from day 10 to day 14.

The cholest-4-en-3-one oxime (10, 30 and 100 mg/kg once daily) was administered by oral route from day 10 to day 14 and the paw withdrawal thresholds were measured 4 hours after administration of the cholest-4-en-3-one oxime. 8 rats were used per tested dose.

On the last day of the treatment (day 14), immediately after the test, plasma samples were obtained from 3 rats of each group treated with the cholest-4-en-3-one oxime or the excipient for pharmacokinetic studies.

The mechanical stimulation thresholds at which the animals withdraw their hind paw in response to mechanical stimulations were measured using a Dynamic Plantar Aesthesiometer (Ugo Basile, Italy). Briefly, in a temperature-controlled room (approximately 22° C.) each animal was placed in a transparent cubic box made of Perspex (22×16.5×14 cm) containing a metal mesh floor giving access to the soles of its paws and left for 15 minutes before the test in order for it to acclimatize. A mechanical stimulus was applied to the left hind paw using a stainless filament (0.5 mm in diameter) exerting a force which increases linearly (2.5 g/sec). The force (g) at which the animal withdraws its paw was recorded automatically. Each paw withdrawal threshold was calculated as the average of three consecutive tests carried out at 5-minute intervals by an investigator ignorant of the treatments used on each of the animals. A 50 g limit was imposed in order to prevent tissue damage.

A stock solution of cholest-4-en-3-one oxime in corn oil was prepared on the first day of the experiment and administered once daily by oral tube feeding (5 ml/kg). The cholest-4-en-3-one oxime was reduced to a fine powder and mixed with the necessary quantity of excipient in order to obtain concentrations of 0.2, 6 and 20 mg/ml, the mixture is then homogenized by magnetic stirring for at least 10 minutes. Stock solutions were kept at 4° C. between administrations. A statistical analysis was carried out using a two-way ANOVA test followed by

Tukey's test in order to compare the effect of the test compound with respect to the excipient.

Results

A tactile allodynia developed in the first two days following the first injection of vincristine (day 3, P=0.015). This was more pronounced on the day after the second injection (day 5, P<0.001) and was totally established after the third injection (day 7, p<0.001). The allodynia remained significant up to 8 days after the last injection of vincristine (day 7-14, P<0.001).

The oral administration of a single dose of cholest-4-en-3-one oxime significantly alleviates the allodynia induced by vincristine at the highest dose (100 mg/kg), in comparison with the animals treated with the excipient (day 10, P<0.001) whereas the lowest doses produce no significant anti-allodynic effect on the first day of the administration (day 10, 10 mg/kg, p=0.086; 30 mg/kg, P=0.232). A daily treatment with cholest-4-en-3-one oxime (10, 30 and 100 mg/kg) from days 11 to 14 (therefore from the second to the last day of administration of cholest-4-en-3-one oxime) significantly alleviates the allodynia induced by the vincristine (P<0.05 in all cases).

### EXAMPLE 2

### Effect of cholest-4-en-3-one oxime on Hypersensitivity to Pain in a Rat Model Presenting Neuropathies Induced by Paclitaxel

Animals

Adult male Sprague-Dawley rats were used.

Paclitaxel (6 mg/ml in a 50:50 mixture of Cremophor and ethanol) was diluted just before use with a 0.9‰ sodium chloride solution at a concentration of 2 mg/ml and injected by intraperitoneal route in a volume of 1 ml/kg on days 0, 2, 4 and 6.

Fresh solutions of cholest-4-en-3 one oxime (Sigma) in corn oil were prepared each week at a concentration of 0.6 or 6.0 mg/ml. This solution was given to the animal by oral tube feeding in a volume of 5 ml/kg.

Three groups of 12 animals treated with paclitaxel were formed:
group 1: cholest-4-en-3-one oxime at 3 mg/kg per os, daily for 17 days (day(−1)-day(15), commencing on the day before the first injection of paclitaxel and continuing for 9 days after the last injection of paclitaxel). On the days when the cholest-4-en-3-one oxime and the paclitaxel had to be administered, the cholest-4-en-3-one oxime was given at 9:00 h, and the paclitaxel at 13:00 h;
group 2: as above except that the cholest-4-en-3-one oxime was given at 30 mg/kg;
group 3: as above except that the animals received an identical volume of excipient (corn oil);

The animals were habituated to the test environment for 3 separate days and then 3 sessions per day were organized in order to establish the normal response to a von Frey hairs (VFH) stimulation exerting pressure of 4 g or 15 g (4 gVFH or 15 gVFH). Normal rats rarely withdraw from a stimulation with 4 gVFH; thus, an increase in response post treatment with paclitaxel is a sign of mechanical allodynia. Normal rats withdraw from a stimulation with 15 gVFH 10-20% of the time; thus an increased frequency of response to this stimulus indicates mechanical hyperalgesia.

The animals were placed in cages containing a metal mesh floor giving access to the soles of the animals' paws. Each mechanical stimulus was applied to the middle of the heel of the left hind paw. The presence or absence of withdrawal of the paw was noted. This was repeated 5 times on each hind paw and the animals' responses were summarized as a percentage of responses (5 withdrawals of the paw in response to a stimulation of 15 gVFH corresponded to a score of 50%).

The rats were habituated to the test environment for 3 days. Tests relating to the reference level of sensitivity at 4 g and 15 gVFH were carried out for 3 consecutive days; the average of the last two corresponded to the level of sensitivity before treatment with paclitaxel. The behaviour tests for testing the effects of paclitaxel commenced on day 16 and were repeated on days 19, 22, 25, 28, 31, 35 and 40, by an investigator ignorant of the treatments applied to each of the animals.

The area-under-the-curve values were calculated for each group during the period from D16 to D40 (from the start of the appearance of the pain due to the paclitaxel to the end of the experiment). The AUC values were analyzed with an ANOVA test followed by comparison “in pairs” with the group having received the excipient by Dunnett's t-test.

Results

The animals treated with paclitaxel which received the excipient significantly developed mechanical allodynia and mechanical hyperalgesia which became apparent on day 16 and persisted for the 40 days of observation.

The 3 mg/kg and 30 mg/kg doses of cholest-4-en-3-one oxime significantly inhibited (p<0.01) the development of mechanical allodynia and hyperalgesia. The amplitude of the effect observed with the two doses was practically identical (59-53% anti-allodynia and 45-40% anti-hyperalgesia for 3 mg/kg and 30 mg/kg respectively). The anti-allodynia and the anti-hyperalgesia were apparent during the first test day (day 16) and persisted throughout the experiment.

### EXAMPLE 3

### Effect of cholest-4-en-3-one oxime on the Neuropathies Induced by Oxaliplatin

The effect of the cholest-4-en-3-one oxime on cold allodynia induced by cumulative doses of oxaliplatin were evaluated in the rat.

Method:

Adult male Sprague-Dawley rats weighing approximately 150 g were used. The oxaliplatin was injected by intra-peritoneal route at 3 mg/kg three times a week and for three weeks (total cumulative dose of 27 mg/kg). The paw withdrawal time as well as the amplitude of the animal's response to harmless thermal stimulations using a drop of acetone were measured in the rats before and after treatment by oxaliplatin on days 1 and 18 respectively, and then on each day of treatment with cholest-4-en-3-one oxime, from day 22 to day 25.

The cholest-4-en-3-one oxime (1, 10 and 100 mg/kg once daily) was administered by oral route from day 22 to day 25 and the thresholds for withdrawal of the paw were measured 4 hours after administration of the cholest-4-en-3-one oxime. 7 to 9 rats were used per dose tested.

On the first and last day of the treatment (day 22 and day 25, respectively), immediately after the test, samples of plasma were obtained from 4 rats of each group treated with cholest-4-en-3-one oxime for pharmacokinetic studies.

For each animal, the reaction times at which the animals withdrew their hind paw following the deposition of the drop of acetone were calculated as the average of six consecutive tests carried out at 5-minute intervals by an investigator ignorant of the treatments applied to each of the animals. A limit of 20 seconds was imposed. The amplitude of the response to the drop of acetone was marked on a scale of 0 to 3: 0 (no response); 1 (rapid withdrawal of the paw); 2 (prolonged withdrawal of the paw); 3 (repeated withdrawal of the paw with licking and/or biting). The score of each of the six tests was totalled.

A stock solution of cholest-4-en-3-one oxime in corn oil was prepared on the first day of the experimentation and administered once daily by oral tube feeding (5 ml/kg). The cholest-4-en-3-one oxime was reduced to a fine powder and mixed with the necessary quantity of vehicle in order to obtain concentrations of 0.2, 2 and 20 mg/ml, the mixture is then homogenized by magnetic stirring for at least 10 minutes. Stock solutions were kept at 4° C. between administrations. A statistical analysis was carried out using a two-way ANOVA test followed by Bonferroni's test in order to compare the effect of the test compound with respect to the vehicle.

Results

After three weeks of oxaliplatin administration, cold allodynia developed (day 18, P<0.001).

The oral administration of a single dose of cholest-4-en-3-one oxime significantly reduced the allodynia induced by oxaliplatin in the highest dose (100 mg/kg), in comparison with the animals treated with the excipient (day 22, P<0.05). The anti-allodynic effect of the cholest-4-en-3-one oxime at a dose of 100 mg/kg persisted after daily treatment from days 22 to 25. The administration of lower doses of cholest-4-en-3-one oxime produced no significant anti-allodynic effect on the first day of administration. Starting from the third day of administration, the lower doses of cholest-4-en-3-one oxime (1 and 10 mg/kg) significantly reduced the cold allodynia induced by oxaliplatin (day 24, P<0.05).

1. A pharmaceutical composition comprising at least one cytotoxic agent, and at least cholest-4-en-3-one oxime or one of its derivatives.

2. The composition according to claim 1, that comprises at least:
a compound of formula I 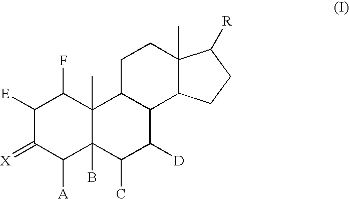
in which
X represents an ═N—OH group,
R represents a group chosen from 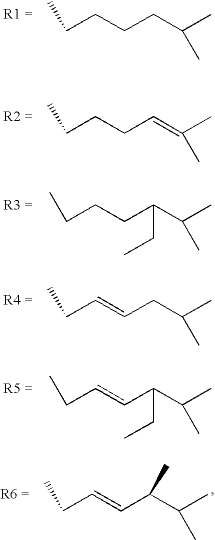
A represents a hydrogen atom or together with B a carbon-carbon bond,
B represents a hydrogen atom, a hydroxy group or together with A a carbon-carbon bond,
C represents a hydrogen atom, a ketone group or an oxime group (═N—OH), or together with D a carbon-carbon bond,
D represents a hydrogen atom or together with C a carbon-carbon bond,
E represents a hydrogen atom or together with F a carbon-carbon bond, or
F represents a hydrogen atom or together with E a carbon-carbon bond, or
an addition salt of said compound of formula I with pharmaceutically acceptable acids, or an ester thereof or an addition salt thereof with pharmaceutically acceptable acids of said ester,
and at least one said cytotoxic agent

3. The composition according to claim 1, wherein for the compound of formula I, when X representing represents an oxime group (═NOH) then:
A represents together with B a carbon-carbon bond; C, D represent a hydrogen atom; E, F represent a hydrogen atom or together a carbon-carbon bond; and R has the meaning R1;
A represents together with B a carbon-carbon bond; C, D represent a hydrogen atom; E, F represent a hydrogen atom; and R has the meaning R2 or R3 or R4;
A represents together with B a double bond; C, represents together with D a carbon-carbon bond; E, F represent a hydrogen atom; and R has the meaning R1 or R6;
A represents together with B a double bond; C represents together with D a carbon-carbon bond; E represents together with F, a carbon-carbon bond; and R has the meaning R1;
E represents together with F a double bond; C, D, A, B represent a hydrogen atom; and R has the meaning R1;
or one of its addition salts with acceptable acids, or one of its esters or one of the addition salts with the acceptable acids of said esters.

4. The composition according to claim 2, wherein the compound of formula I is chosen from cholestan-3-one oxime, cholest-4-en-3-one oxime, or cholest-1,4-dien-3-one oxime, or one of their addition salts with the pharmaceutically acceptable acids, or one of their esters, or one of the esters of the addition salts.

5. The composition according to claim 2, wherein the compound of formula I is cholest-4-en-3-one oxime.

6. The composition according to claim 1, wherein the cytotoxic agent is an anticancer cytotoxic agent and/or an agent inducing in a patient symptoms of peripheral neuropathies and/or damage to the nervous system.

7. The composition according to claim 6, wherein the cytotoxic agent is chosen from cisplatin or its derivatives, periwinkle alkaloids (vinca) or the taxanes.

8. The composition according to claim 7, wherein the cisplatin derivative is chosen from carboplatin or oxaliplatin.

9. The composition according to claim 7, wherein the periwinkle alkaloid is chosen from vinblastine, vincristine, vindesine or vinorelbine.

10. The composition according to claim 7, wherein the taxane is chosen from docetaxel and paclitaxel.

11. The composition according to claim 7, wherein the cytotoxic agent is chosen from oxaliplatin, carboplatin, cisplatin, vinblastine, vincristine or paclitaxel.

12. The composition according to claim 11, wherein the cytotoxic agent is chosen from oxaliplatin, vincristine or paclitaxel.

13. The composition according to claim 1, wherein the cytotoxic agent and/or cholest-4-en-3-one oxime or one of its derivatives are each in a quantity comprised between 0.1 and 2000 mg/ml.

14. The composition according to claim 1, wherein the cytotoxic agent and/or cholest-4-en-3-one oxime or one of its derivatives are in a cytotoxic agent/cholest-4-en-3-one oxime derivatives ratio comprised between 0.1 and 300.

15. The composition according to claim 1, wherein the cytotoxic agent and/or cholest-4-en-3-one oxime or one of its derivatives are each in a quantity comprised between 0.1 and 2000 mg/ml and in an anticancer compound/cholest-4-en-3-one oxime or derivatives ratio comprised between 0.1 and 300.

16. The composition according to claim 1 that comprises cholest-4-en-3-one oxime and vincristine.

17. The composition according to claim 1 it that comprises cholest-4-en-3-one oxime and oxiplatin.

18. The composition according to claim 1 it that comprises cholest-4-en-3-one oxime and paclitaxel.

19. The composition according to claim 1, wherein the compounds of said composition are in an identical or different galenic form.

20. A method to treat cancer while preventing and/or treating the side effects of peripheral neuropathy type caused by the anticancer treatment, comprising administering the pharmaceutical composition of claim 1 to a patient in need thereof.

21. A method to prevent and/or treat the side effects of peripheral neuropathy type caused an the anticancer treatment, comprising administering the pharmaceutical composition of claim 1, to a patient in need thereof.

22. A method to treat cancer while preventing and/or treating the side effects of peripheral neuropathy type caused by an anticancer treatment, comprising administering the pharmaceutical composition of claim 5 to a patient in need thereof.

23. A method to prevent and/or treat the side effects of peripheral neuropathy type caused by an anticancer treatment, comprising administering the pharmaceutical composition of claim 5, to a patient in need thereof.
